(19) United States
(12) Patent Application Publication
(10) Pub. No.: US 2022/0118090 A1
(43) Pub. Date: Apr. 21, 2022

(54) METHOD AND DEVICE FOR TREATING PARTICLES AND NANOPARTICLES OF AN ACTIVE PHARMACEUTICAL INGREDIENT

(71) Applicant: TRUMPF Laser- und Systemtechnik GmbH, Ditzingen (DE)

(72) Inventors: Marcus Lau, Stuttgart (DE); Andreas Popp, Markgroeningen (DE)

(21) Appl. No.: 17/430,451

(22) PCT Filed: Feb. 12, 2020
(86) PCT No.: PCT/EP2020/053553

§ 371 Date: Aug. 12, 2021

(30) Foreign Application Priority Data
Feb. 12, 2019 (DE) 102019201781.9
Jan. 20, 2020 (DE) 102020101164.4

(51) Int. Cl. A61K 41/00; A61K 9/19

(52) U.S. Cl. CPC A61K 41/00; A61K 9/19

(57) ABSTRACT

A method and a device for treating particles and nanoparticles in a suspension. A liquid jet is generated in which the particles are entrained. The liquid jet is irradiated with at least two laser beams, preferably pulsed beams, from mutually different directions. The particles are thereby comminuted. The suspension is analyzed before and/or after the irradiation. The liquid of the liquid jet is then collected in a collection vessel.

The invention relates to a method for treating particles, in particular micro- and nanoparticles, in a suspension. The invention also relates to a device for treating such particles. The invention also relates to nanoparticles of an active pharmaceutical ingredient.

Particles with sizes in the micro or nanometer range have a wide range of applications in a wide variety of fields of technology. The production of particles of certain sizes and size distributions can be important here. The size, shape and/or texture, for example a surface or a crystal structure, of the particles can also be important properties of the particles.

Traditionally, colloid mills are used to crush particles with sizes in the micro or nanometer range used. Such mechanical comminution devices are sometimes subject to high wear and tear, and their use also means that particles from the material of the mill get into the suspension.

The time-shifted irradiation of a suspension with a plurality of lasers is known from EP 2 735 390 A1. In this case, particles are generated in an aqueous medium by irradiating a substrate with a first laser. A beam of the aqueous medium with the particles then irradiated by a second laser in order to fragment or comminute the particles.

The object of the invention is to provide a method and a device for the efficient, in particular reproducible and in particular post-controllable treatment, in particular comminution, of particles in a liquid jet. Furthermore, the object of the invention is to create nanoparticles of an active pharmaceutical ingredient that are free of contamination and have a traceable manufacturing history.

This object is achieved according to the invention by a method according to claim 1, a device according to claim 12 and nanoparticles according to claim 19.

The method according to the invention relates in particular to the treatment of particles which in the initial state have a size in the micrometer, submicrometer and nanometer range. In particular, the size of the particles can be less than 0.1 mm, in particular less than 0.01 mm, and more than 1 nm, in particular 500 nm. It is in particular in accordance with the invention if a device according to the invention is used to carry out the method according to the invention.

The method according to the invention for treating particles comprises the steps of:
a) generating a liquid jet. The particles are entrained in the liquid jet. The particles are present in the liquid jet in a suspension.
The liquid jet can be guided in a guide structure, for example a channel, pipe or hose. The guiding structure is transparent to the laser beam used, at least in portions. However, the liquid jet can also be a freely falling liquid jet. A freely falling liquid jet is understood here to mean a liquid jet that is not guided. In particular, this is understood to mean a liquid jet falling downward freely (in particular in a straight line) under the influence of gravity. The liquid jet can emerge from a jet generating device, for example a nozzle, which is used to generate the jet with or without pressure. A liquid jet here in particular means a continuous liquid column and not a sequence of individual drops.
b) Irradiating the liquid jet having at least two laser beams from different directions in each case.
The irradiation by means of a plurality of laser beams from different directions serves to irradiate as many regions as possible in the cross section of the liquid jet. In particular, it is provided that the lasers are arranged so that they are directed at the liquid jet in such a way that no portions remain in the cross section that are not covered by laser radiation. In other words, the laser beams are directed onto the liquid jet in such a way that all portions of the cross section of the liquid jet are captured by the laser beams. This is described in more detail below. This means that all entrained particles can be treated with the laser beams in one pass. It is in particular in accordance with the invention that pulsed laser beams (in particular pulse duration of the laser beams in the picosecond range, femtosecond range or nanosecond range) are used. In particular, the irradiation with the lasers serves to comminute the particles. The irradiation runs without wear and, in contrast to mechanical treatment or comminution, without contamination of the particles or the suspension. Wavelengths that interact sufficiently with the particles in order to efficiently comminute them are particularly suitable here. In addition, high repetition rates of the laser pulses are advantageous. The wavelength of the laser beams can in particular be, for example, 532 nm or 1030 nm or 515 nm or 343 nm, wherein it is also possible to use a plurality of laser beams having different wavelengths. In particular, it can be provided to use Yb:YAG lasers.
c) Analysis of the suspension before and/or after the irradiation by means of the laser beams.
The method according to the invention therefore provides that the suspension in which the particles are contained is analyzed. This analysis can be done before or after the irradiation. In particular, the suspension is to be analyzed before and after the irradiation. The analysis can be used to control the irradiation process. In particular, the storage of the analysis results is provided. In an analysis that is carried out before the irradiation, it can be checked whether the particles fed in have the appropriate initial size. It is also possible to adapt the parameters of the irradiation in step b) based on the analysis results of the analysis before the irradiation. In an analysis that follows the irradiation, the result of the irradiation, in particular the comminution of the particles, can be checked. Carrying out the analysis before and after the irradiation process can, for example, (also) be used to check whether there are any disturbances in the irradiation process. It is also possible to adapt the parameters of the irradiation in step b) based on the analysis results of the analysis after the irradiation, for example until a target value is reached.
d) Collect the liquid from the liquid jet in a collection vessel. This can mean actually collecting an unguided, freely falling liquid jet. However, collecting in a collection vessel can also mean the introduction of a guided liquid jet.
Steps c) and d) can be provided alternatively or jointly.

The particles can comprise inorganic material or consist of inorganic material. In particular, the material can be metal, for example gold or platinum. Such particles can be used, for example, as catalysts.

The use of the method according to the invention or the use of the device according to the invention for treating particles from active pharmaceutical ingredients (or particles comprising active pharmaceutical ingredients), in particular poorly water-soluble active pharmaceutical ingredients, is also within the meaning of the invention. Active ingredients that are suitable to be treated or comminuted with the method according to the invention are, for example:

ampicillin, benzylpenicillin-benzathine, benzylpenicillin-procaine, cefazolin, ceftazidime, imipenem, chloramphenicol, ciprofloxacin, phenobarbital, phenytoin, metronidazole, trimethoprim, sulfamethoxazole, linezolid, paraaminosalicylic acid, amphotericin B, fluconazole, 5-fluorcytosine, aciclovir, quinine, melarsoprol, azathioprine, cyclosporine, folinic acid, carboplatin, dacarbazine, actinomycin D, daunorubicin, docetaxel, etoposide, ifosfamide, paclitaxel, cortisol, methylprednisolone, biperiden, digoxin, adrenaline, lidocaine, verapamil, amiodarone, digoxin, furosemide, selenium sulfide, fluorescein, tropicamide, dexamethasone, ondansetron, testosterone, medroxyprogesterone, estradiol-17-beta-cypionate, glucagon, azithromycin, ofloxacin, tetracycline, prednisolone, timolol, atropine, ergometrine, in general: ergot alkaloids, for example LSD and methylergometrine, fluphenazine, risperidone, clozapine, fluoxetine, carbamazepine, diazepam, beclomethasone dipropionate, budesonide, ipratropium bromide, salbutamol, budesonide, chloroquine, penicillamine, ketoconazole, fenofibrate, naproxen.

A separate independent invention is also constituted by nanoparticles of an active pharmaceutical ingredient, in particular of one or more of the aforementioned active ingredients, which were comminuted by means of steps a) and b) and were analyzed by means of step c). The fact that she particles were created by steps a) and b) can be seen from the particles, for example, by the fact that they have no impurities and a narrow size spectrum, which is achieved by irradiating the entire liquid jet cross section by means of the laser from different directions The analysis result (analysis step c)) is assigned to the nanoparticles. This means that the analysis result can be reproduced and there is a connection to the nanoparticles characterized by this analysis result, so that it can be clearly identified which particles are described or “measured” by the corresponding analysis. The independent invention of the nanoparticles will be discussed in detail below.

In particular, it is provided that the material of the particles can have a solubility in the liquid (the suspension) of less than 10 g/L, in particular less than 1 g/L, as a liquid a physiological solution can in particular be provided. The particles can be present in dispersed form in the physiological solution.

According to the invention, in the case of the method and in the case of the particles, it can be provided that the particles are or have been dispersed in the liquid by means of an auxiliary substance in the initial state, that is to say before irradiation. For this purpose, the suspension can contain an additive such as cellulose, hydroxyethyl cellulose, polyvinyl alcohol (PVA) , sodium dodecyl sulfate (SDS) , polyvinylpyrrolidone (PVP), polysorbate 80, sodium citrate and phosphate buffer for particle stabilization. The presence of such an additive can also be recognized by the particles or the suspension in which the particles can be present.

For Laser Irradiation in Step b) or for the Formation of the Laser Assembly in the Corresponding Device:

It can be provided that the liquid jet is irradiated in step b) with at least three laser beams from each of the different directions. As a result, the liquid jet is reliably exposed to the laser radiation. Or a particularly uniform intensity of the laser radiation within the liquid jet is ensured. The (two, three or more) laser beams can be rotationally symmetrical with respect to the liquid jet.

The laser beams are preferably pulsed laser beams. This can increase the effectiveness of the treatment. A pulse repetition rate of the laser pulses is typically matched to the flow velocity of the liquid jet in such a way that all partial volumes of she liquid jet are hit by at least one laser pulse of all laser beams.

The laser beams can hit the liquid jet offset from one another in the flow direction of the liquid jet. In particular, however, it can be provided that at least two laser beams, in particular all laser beams, hit the liquid jet at the same height in the flow direction of the liquid jet. In other words, the laser beams hit the liquid jet in the flow direction at the same point, i.e. in a common area of incidence. This increases the energy acting on the particles captured by the laser beams and ensures that no liquid volumes escape irradiation due to fluid-mechanical influences. In the case of pulsed laser beams, the individual pulses of the plurality of laser beams hit the liquid jet preferably at the same time or at least essentially at the same time.

Essentially simultaneous impingement means that a time offset of the impingement of the pulses of the plurality of laser beams is so small that the particles do not cover any significant distances in the flow direction of the liquid jet in this time interval. Paths that are smaller, in particular one or more orders of magnitude smaller, than a length (measured in the flow direction of the liquid jet) of the laser-liquid interaction zone can be regarded as insignificant distances. In other words, the timing of the pulses can be matched to the flow velocity so that no volume of liquid passes the area of incidence without being irradiated by a laser pulse.

The laser beams preferably run in a common plane which, in particular, is oriented perpendicular to the liquid jet. This can further increase the effectiveness of the treatment. In particular, diffraction effects when the laser beams hit the liquid jet can be avoided or at least reduced. It is generally provided that the laser beams hit the liquid jet at angles or, in particular, at right angles to the direction of flow. Diffraction and/or reflection effects can be reduced or avoided in particular when they hit at right angles.

The lasers of the laser assembly or those used when carrying out the method can in particular be directed at an angle to the flow direction of the liquid jet that is smaller than or equal to the Brewster angle. It can be provided that, depending on the type of radiation used and the optical properties of the phase boundaries between the liquid jet and the surrounding air, an angle of incidence is selected at which the reflection is minimized when the laser beam hits the liquid jet, and the transmission at the phase boundary when the laser beam passes through between the liquid jet and the surrounding air is minimized when it emerges. By using the internal reflections of the phase boundary, as much laser energy as possible can be kept or used in the liquid jet, while at the same time the reflection on entry is minimized.

As already described, the particles can be comminuted (fragmented) in step b). The method according to the invention or its step b) can be carried out a plurality of times in order to obtain even smaller particles or to improve their size distribution.

In the method according to the invention and the device according to the invention, the pulse duration of the laser beams (in particular to cause the particles to be comminuted) can be in the picosecond range, i.e. at least one picosecond, in particular less than 100 picoseconds, in particular a few hundred picoseconds, but the pulse duration can also be more than a nanosecond (short-pulsed and ultra-short-pulsed laser radiation). A wavelength of the laser beams, in particular with a pulse duration in the picosecond range, can be at least. 500 nm, preferably at least 520 nm, particularly preferably at least 530 nm, and/or the wavelength of the laser beams can be at most 560 nm, preferably at most 540 nm, particularly preferably at most 535 nm. The wavelength of the laser beams can in particular, for example, be 532 nm or 1030 nm or 515 nm or 343 nm, it also being possible to use a plurality of laser beams with different wavelengths. In particular, it can be provided to use Yb:YAG lasers.

In a variant of the invention that is alternative but also advantageous, the particles can be remelted and/or fused in step b) or in the device. When the particles are remelted, at least a partial region of the surface of the particles is melted and, after the particles have solidified, a different shape of the particles and/or a different surface structure is obtained. In other words, the particles can be reshaped, in particular in order to obtain particularly round (spherical) particles. Furthermore, defects, in particular in the surface of the particles, can be generated in a targeted manner. In the fusion, a plurality of particles are joined to one another. In this way, particles with special properties can be obtained. Particles can also be produced from hybrid materials. A chemical conversion of the particles can take place. The pulse duration of the laser beams (especially for remelting) can be in the nanosecond range, i.e. at least one nanosecond and less than one microsecond (short-pulsed laser radiation). A wavelength of the laser beams (in particular for remelting or in particular with a pulse duration in the nanosecond range) can be an most 380 nm, preferably at most 360 nm, particularly preferably at most 350 nm, and/or the wavelength of the laser beams can be at least 310 nm, preferably at least 330 nm, particularly preferably at least 340 nm. In particular, the wavelength of the laser beams is 343 nm.

The device can be designed in such a way that the laser beams in the area of incidence have a width which exceeds the diameter of the liquid jet. This also applies to the method. The device can, for example, have a focusing device for each laser beam via which the laser beam can be focused or its width can be adjusted.

For the Analysis in Step c) or the Analysis Device and the Analysis Result:

The analysis of the suspension includes, in particular, a particle size measurement. The maximum particle size can be determined here. It is also conceivable to determine the minimum particle size. In particular, however, it is provided that a size distribution of the particles is measured.

In the context of the present invention, it can be provided in particular that the device comprises an analysis device which is designed to carry out a (in particular on-line or off-line) measurement by means of dynamic light scattering (DLS) or laser diffraction. The method can comprise a corresponding analysis step c). By scattering the particles in the suspension, it is possible to determine their size distribution. Measurements can be carried out in a short period of time by means of dynamic light scattering, wherein this method is particularly suitable for narrow size distributions, as can be achieved with the present device or the method, in particular when the size distribution has only one mode.

In the context of the present invention, it can be provided in particular that the device comprises an analysis device which is designed to carry out an off-line measurement by means of an analytical disc centrifuge. The method can comprise a corresponding analysis step c). This analysis can be used for the analysis of single- or multi-modal particle size distributions. Particles with a tendency to agglomerate can also be measured with this method.

In the context of the present invention, it can be provided in particular that the device comprises an analysis device which is designed to carry out a measurement by means of ultrasonic extinction. The measurement can be carried out in-line, on-line or even off-line. The method can comprise a corresponding analysis step c). As a result, in particular a particle size distribution can be recorded in-line, which can be used, for example, for a rapid correction of process parameters if deviations from a target value are recorded.

According to the invention, it can also be provided that within the scope of the method x-ray diffraction measurement is carried out to determine the crystal structure of the particles or a corresponding analysis device can be provided for the device. In particular, it can be provided that the crystal structure of the particles is determined or stored and/or compared (for example to reference values, for example from previous measurements) based on characteristic values obtained from the analysis. The corresponding measurement is typically carried out as off-line measurement or the analysis device is configured for an off-line measurement. In particular, it is provided that the corresponding analysis is carried out on particles in the dried state. For this purpose, the method can comprise a drying step preceding the analysis (for example spray-drying or freeze-drying step) or the device can comprise a corresponding drying device.

According to the invention, it can also be provided that a spectroscopic measurement is carried out in analysis step c) as part of the method. In the context of the present invention, it is provided in particular that the device comprises an analysis device which is designed to carry out an on-line or in-line spectroscopy measurement (e.g. spectroscopy in the UV, VIS, NIR or MIR range is conceivable), or it is provided that the method includes a corresponding analysis step c). This provides a fast analysis method by means of which the suspension can be analyzed directly. In particular, this variant does not require a sample to be taken which must then be disposed of, thereby making the method more efficient.

In the context of the present invention, it can be provided in particular that the device comprises an analysis device which is provided for performing an off-line measurement by means of x-ray photoelectron spectroscopy (XPS). The method can comprise a corresponding analysis step c). This allows a chemical analysis of the surface of the particles. In particular, it is provided that the corresponding analysis is carried out on particles in the dried state. For this purpose, the method can comprise a drying step preceding the analysis (for example spray-drying or freeze-drying step (lyophilization)) or the device can comprise a corresponding drying device.

In the context of the present invention, it can be provided in particular that the device comprises an analysis device which is designed to carry out an off-line measurement by means of nuclear magnetic resonance spectroscopy (NMR spectroscopy), for example to identify chemical bonds of the particles. The method can comprise a corresponding analysis step c).

According to the invention it can also be provided that within the scope of the method a chromatographic (especially HPLC, that is high performance liquid chromatography) measurement is carried out or the device comprises a correspondingly configured analysis device. The corresponding analysis device is typically designed to carry out an off-line measurement by means of high performance liquid chromatography (HPLC). It is also conceivable to use this analysis method as an in-line measurement, for example via a fluidic bypass, for example via the flow divider described in this application. A chromatographic separation can also be provided for purification or particle size selection.

An in-line or on-line pH value and/or temperature measurement can also be provided.

It is also possible to use a plurality of the types of analysis or analysis devices just mentioned in combination or to provide them in the device according to the invention.

According to the invention, it can be provided that an analysis is carried out before and after the irradiation, by means of which the same measured variable (as already described above, e.g. maximum or minimum particle size, particle size distribution or crystal structure) is recorded, in particular wherein the same measurement method is used.

According to the invention, it can be provided that the liquid jet is separated into batches or is collected batchwise. This also means if the liquid jet is interrupted between individual batches. According to the invention, an analysis result can be assigned to each batch as part of the method.

According to the invention, the result of the analysis can be stored in a database. In particular, an assigned analysis result can be stored in the database for each batch as part of the method.

In the context of the method according to the invention, it can be provided that the analysis includes an on-line and/or an in-line measurement. That therefore continuous in any case a part of the liquid of the liquid jet is analyzed or the liquid jet itself is analyzed in real time. For this purpose, for example, the freely falling beam can be subjected to an analysis or measurement before or after the irradiation. It is also conceivable that the beam is captured and fed to an on-line measurement via a line. The liquid of the liquid jet can also be subjected to an analysis or measurement before the irradiation is carried out and before it is fed to the jet generating device.

In the context of the method according to the invention, it can be provided that the analysis includes a batch measurement, wherein in particular a batchwise measurement is carried out for each batch. This means that a measurement is carried out for each batch. This can be carried out “on-line” during the treatment of the batch or “off-line” in such a way that, in contrast to the measurements or analyses carried out on-line, the batch is first collected and then a sample is taken which is subjected to an analysis or the entire batch is analyzed. For an analysis of the entire batch, a “contactless” analysis method (for example an optical measuring method) is preferably used, as a result of which the risk of contamination can be reduced.

In the context of the method according to the invention, it can be provided that the liquid of the liquid jet is divided into a main flow and a secondary flow. Many analytical methods only require a small amount of liquid. Accordingly, the secondary flow can be fed to the analysis device, and the main flow can already be treated processed. It is also conceivable that the secondary flow is mixed again with the main flow following the analysis. The division into a main stream and a secondary flow is particularly useful for performing on-line analyses.

The analysis results can be saved continuously in digital form. In particular, the method can include a comparison, in particular taking place quasi in real time, of newly determined analysis results to analysis results already stored or to other reference values. Provision can be made for process parameters to be adjusted based on this comparison. For example, parameters of the laser radiation, such as the pulse duration or the intensity, can be adapted based on the comparison. It is conceivable, for example, that a certain maximum particle size is specified and analysis results are continuously compared to this target size and the parameters of the laser radiation are adapted until the analysis results match the target size.

It is also conceivable that the analysis results are transmitted to a database of, for example, an official authority such as the European Chemicals Agency (ECHA). In particular, the transmission can be carried out in batches.

The analysis results can be stored in at least one blockchain within the scope of the invention. This allows forgery-proof continuous storage of the analysis results. In particular, the blockchain can continue to be written for a further batch. In this context, a blockchain is understood to mean a database whose integrity, i.e. protection against subsequent manipulation, is secured by storing a hash value of a previous data record in the subsequent data record, i.e. by cryptographic chaining. Exactly one blockchain can be provided. A plurality of blockchains can also be provided. In particular, provision can be made for a new data record to be created in the blockchain for each batch. The blockchain can be stored and processed in a distributed computing system. A central computing system can also be provided. Access rights to information from the blockchain can be configurable. Access to the blockchain can be restricted. For example, a cryptographic key is used for this purpose which allows a subscriber to transmit the status changes in encrypted form and thus prevents the statuses from being read by unauthorized persons. This encryption can be chosen so that it does not affect the headers, which in this case are transmitted unencrypted in order to allow the verification of a data record. Cryptographic key pairs can give one or more participants in the blockchain targeted access to encrypted data records without these generally becoming accessible to all other participants in the blockchain. It can also be provided that the method according to the invention is carried out on a plurality of corresponding devices and that these devices each write their analysis results in a common blockchain. Correspondingly, a plurality of devices according to the invention can be designed to be networked with one another in such a way that they can write their analysis results to a common blockchain.

Regarding the Device:

The device according to the invention comprises:
On the one hand, a jet generating device for generating a liquid jet loaded with particles. The jet generating device can be designed as a nozzle, for example. The jet generating device can be designed in order to be able to adjust the diameter of the liquid jet. In particular, it can be provided that the radiation generating device generates an unguided liquid jet. The device is preferably designed in such a way that the unguided liquid jet is generated in a freely falling manner, in particular in a straight line.
also a laser assembly. The laser assembly is designed in such a way that it serves to generate at least two laser beams. In particular, the laser beams can be pulsed. For this purpose, reference is made to the statements made above regarding pulsed laser beams. The laser assembly can be designed in accordance with the statements made there. The laser assembly is designed in such a way that it directs two laser beams onto the liquid jet, wherein the laser beams hit the liquid jet in different directions. The corresponding advantages in connection with the avoidance of unirradiated portions of the liquid jet have already been explained in connection with the method.
further, a collection vessel which is designed and arranged to collect the liquid of the irradiated liquid jet.
and an analysis device in addition or as an alternative to the collection vessel. The analysis device is designed and arranged in the device in such a way that the suspension of the liquid jet can be analyzed by means of the analysis device. This analysis can be done before or after the irradiation. For this purpose, the analysis device is correspondingly integrated into the device, for example connected via appropriate fluidic connections. It is also conceivable that an analysis can be carried out by means of the analysis device both before and after the irradiation. For this purpose, the analysis device can comprise separate respective measuring devices for the analysis upstream and downstream of the irradiation or use the same measuring device for both analyses.

The device can further comprise an enclosure which is impermeable to the laser radiation of the laser beams. The housing surrounds an area of incidence of the laser beams on the liquid jet in order to prevent laser radiation from escaping and to increase the operational safety of the device.

The device can comprise a reflection housing. The reflection housing is arranged in particular around the area of incidence, in particular around the entire circumference. The reflection housing has a reflective inner surface, that is to say the surface facing the liquid jet. The (inner, liquid-jet-facing) surface is designed and arranged in particular in such a way that it reflects laser radiation that passes through the liquid jet back into the liquid jet. For example, the inner surface of the reflection housing can be designed to be circular and arranged concentrically to the liquid jet. It can be provided that the reflection housing has a plurality of lenses (for example cylinder lenses) for coupling the individual laser beams into the reflection housing. The lenses are typically arranged and designed in such a way that they continue to direct the laser beam onto the liquid jet in the same direction in which it hit the lens.

On the one hand, the reflection housing prevents laser radiation from escaping from the area of incidence as a result of the liquid jet, thereby increasing operational reliability. On the other hand, better use is made of the laser radiation used, because radiation that has passed through the liquid jet is reflected back through the reflection housing and directed back onto the liquid jet.

The device preferably also has at least one power measuring device for measuring a residual power of at least one of the laser beams on the other side of the liquid jet. The degree of absorption when the laser beam or the laser beams hit the liquid jet can be determined from the residual power (with a known output power). The degree of absorption can be used to control the device for effective treatment of the particles, for example for power regulation of the laser assembly

The laser assembly is preferably designed in such a way that the laser beams run in a common plane. The plane can in particular be aligned perpendicular to the liquid jet. The laser assembly can comprise at least two, preferably three, lasers (in the sense of laser beam sources). However, the laser assembly can also have precisely one laser and one beam splitter device for generating the at least two laser beams and at least two light guide devices for guiding the at least two laser beams. See, for example, the above explanations on irradiation.

Typically, the device is configured in such a way that all laser beams are designed in the same way. In particular, all of the laser beams preferably have the same wavelength. In the case of pulsed laser beams, the same pulse durations and pulse repetition rates are typically configured. The light pulses of the laser beams hit the liquid jet preferably synchronously with one another. The pulse energy of the individual laser beams can be identical. Alternatively, at least one of the laser beams can have a different pulse energy. The device can be configured to carry out the different types or further development of step b).

The analysis device can comprise a particle size measuring device. The maximum or minimum particle size or the size distribution of the particles before or after the irradiation can be of particular interest.

The analysis device can comprise an x-ray diffraction measurement device. It can be of interest no characterize the crystal structure of the particles. In particular, it can be of interest to detect a change in the crystal structure. For this purpose, a corresponding measurement can be carried out before and after the irradiation and then the measurement results can be compared.

The analysis device can further comprise a chromatographic measuring device. For example, the molecular structure of the particles can be of interest. In particular, it can be of interest to check whether the irradiation has caused a change in the chemical composition of the particles

In further developments, the analysis device can comprise a measuring device for performing spectroscopic measurements (e.g. photoelectron spectroscopy. Fourier transform infrared spectroscopy and/or UV/VIS spectroscopy). The analysis device can comprise a measuring device for carrying out particle size analysis (for example by means of dynamic light scattering, analytical centrifugation or laser diffraction). The analysis device can comprise a measuring device for carrying out a crystal analysis (for example by means of x-ray diffraction) or a chromatographic measurement (for example HPLC).

The device can comprise a flow divider device. The flow divider device is designed to divide the liquid of the liquid jet into a main flow and a secondary flow. The device can be configured in such a way that the secondary flow is fed to the analysis device. In particular it can be provided that the secondary flow is merged or mixed together again with the main flow after the analysis and that the device has a corresponding line routing. Such a flow divider device can be provided before and/or after the beam generating device.

The device can further comprise a portioning device. By means of the portioning device, it is possible to portion the liquid jet or its liquid in batches, so that the individual batches are fluidically separated from one another. The portioning device can be coupled with the aforementioned analysis and detection methods in such a way that automated portioning takes place as soon as the suspension and/or particle properties move outside of predetermined target values.

It can further be provided that the device comprises a filling device by means of which a certain amount of liquid that has already been subjected to the irradiation can be transferred into a vessel. The device can furthermore comprise an identification device which can produce a relationship between a certain amount of liquid (for example a batch or part of a batch), which for example can be filled in a vessel, and a data set containing analysis results for this amount of liquids.

It can further be provided that the device comprises an extraction device which is designed to extract sample volumes of the liquid from the liquid jet. The extraction device can be arranged upstream and (for example two extraction devices)/or downstream of the beam generating device in the direction of flow. The extraction device can be configured for manual sampling, for example. Also conceivable is automated sampling, which, for example, carries out an automated sampling and delivery to the analysis device at certain time intervals or in response to a user-based control pulse.

It can also be provided that the device comprises a sterile filtration device. The still filtration device allows aseptic dispensing of the liquid from she liquid jet into a vessel. It can be advantageous if the vessel can be sealed. As a result, the particle suspension can be filtered and dispensed directly after the treatment of the particles and, if the vessel is sealable, it can then be sealed and the particles can be stored or delivered in an unpolluted state.

The device can also comprise a spray-drying or freeze-drying device. This allows the particles present in suspension to be easily converted into powder form. The integration of such a drying device into the device offers the advantage of a closed process chain in a single device. In particular, this can reduce the risk of contamination during processing of the particles.

Correspondingly, the method according to the invention can also comprise a sterile filtration step or a spray drying or freeze-drying step.

Regarding the Nanoparticles:

As already mentioned at the beginning, nanoparticles of an active pharmaceutical ingredient represent an independent part of the present invention. The nanoparticles were comminuted using steps a) and b) and analyzed using step c). That analysis result lies in a form assignable to the nanoparticles. This can be used to provide evidence of quality, for example. The analysis result can be available locally; alternatively, in particular additionally, it can also be transmitted to an external database and stored there, for example in the database of an authority.

The nanoparticles can be in the form of a batch that was fragmented under uniform process conditions in step b). The nanoparticles can be assigned a data record that includes the analysis result. In addition, the data record can include an operating parameter characteristic for the irradiation in step b).

The batch of nanoparticles can be present in a mechanically manageable vessel. The vessel can in particular comprise a machine-readable identification feature (for example a QR code). In this way, the assignment of she batch to the data record or the analysis result can be simplified.

The nanoparticles can be present in a suspension in an aqueous medium. The suspension can further comprise an additive for particle stabilization. In the context of the invention, cellulose, polyvinyl alcohol, polyvinylpyrrolidone (PVP), sodium dodecyl sulfate (SDS), polysorbate 80 or another surface- and/or interface-active substance is provided as an additive, as already mentioned at the beginning. The type and/or concentration of the additive can be contained in the data set. This means that a manageable number of- nanoparticles is available, wherein the properties of the suspension liquid and the parameters for producing the nanoparticles are present in a directly available and comprehensible form.

The particles can also have been converted into powder form using a spray drying or freeze-drying step. The powdery particles can then have been transferred to a vessel, as described above.

Further aspects and details of the present invention are illustrated in the accompanying drawing and described in more detail using embodiments:

### IN THE DRAWINGS

FIG. 1 is a cross section through a liquid jet when treating with a method according to the prior art;

FIG. 2 shows a device according to the invention having two lasers in a schematic side view;

FIG. 3 shows a laser assembly having three lasers during the treatment of particles in a liquid jet, in a schematic plan view;

FIG. 4 is a schematic representation of the incidence of the laser beams on the liquid jet during treatment of the particles with the laser assembly of FIG. 3;

FIG. 5 is a flowchart of a method according to the invention for treating particles;

FIG. 6 shows a device according to the invention with two lasers in a schematic side view;

FIG. 7 shows a further laser assembly with three lasers during the treatment of particles in a liquid jet, in a schematic plan view;

FIG. 8 shows a further laser assembly with three lasers during the treatment of particles in a liquid jet, in a schematic plan view;

FIG. 9 is a further flowchart of a method according to the invention for treating particles;

FIG. 10 is a schematic representation of an area of incidence when looking along a liquid jet;

FIG. 11 is a schematic representation of an area of incidence when looking orthogonal to the liquid jet;

FIG. 12 is a schematic representation of an area of incidence when looking along a liquid jet;

FIG. 13 shows a further laser assembly with two lasers during the treatment of particles in a liquid jet in a schematic plan view; and

FIG. 14 shows a further laser assembly with a laser in the treatment of particles in a liquid jet in a schematic plan view.

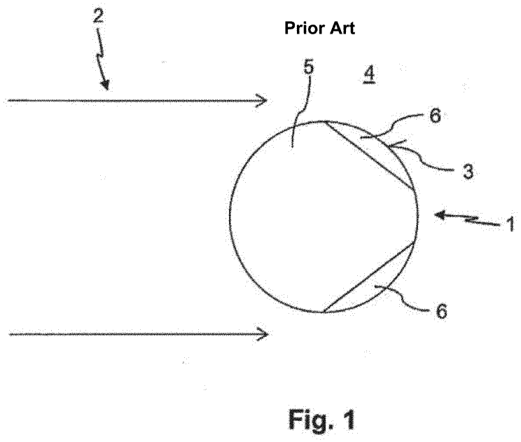

FIG. 1 shows a cross section through a liquid jet 1 with particles (not shown) during treatment with a single laser beam 2 according to the prior art as per EP 2 735 390 A1.

The laser beam 2, coming from the beam direction, detects the liquid jet 1 over its entire width. However, the laser radiation is diffracted when it hits the interface 3 between the liquid jet 1 and the environment 4 (air). As a result of the diffraction, in addition to an irradiated portion 5, portions 6 also arise within the cross section of the liquid jet 1 which are not reached by the laser beam 2. Particles located in these non-captured portions 6 are therefore not hit by the laser radiation and cannot be treated.

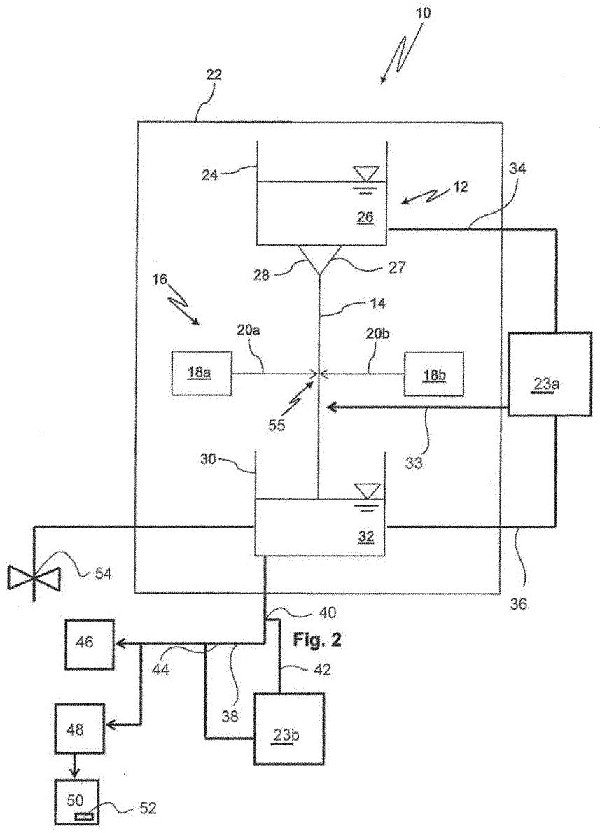

FIG. 2 shows a device 10 according to the invention for treating particles. The device 10 includes a beam generating device 12 for generating a liquid jet 14 loaded with particles. The device 10 further comprises a laser assembly 16 with two lasers 18a, 18b, The lasers 18a, 18b emit pulsed laser beams 20a, 20b, The laser beams 20a, 20b are directed onto the liquid jet 11 from opposite directions. The laser assembly 16 and the beam generating device 12 are arranged as a whole within a housing 22. The housing 22 is impermeable to the laser radiation of the laser beams 20a, 20b. The device 10 further comprises a first analysis device 23a and a second analysis device 23b.

The jet generating device 12 comprises a storage vessel 24 in which a liquid 26, in this case an aqueous liquid 26, with particles (riot shown) suspended therein is stored. A jet generating device 27 with a nozzle 28 is arranged on the storage vessel 24.

The jet generating device 27 or nozzle 28 allows the liquid 26 with the particles to exit from the storage vessel 24, so that the liquid jet 14 is created. The nozzle 28 works here without pressure (apart from the back pressure of the liquid in the storage vessel 24). In an alternative not shown, the nozzle 28 could be connected to a pump of the jet generating device 27, which would allow the liquid 26 to exit the nozzle 28 under pressure. After exiting the nozzle 28, the liquid jet 14 falls freely (unguided) under the influence of gravity in a straight line downward. Here, different geometries of the nozzle are conceivable which can advantageously lead to changes in shape of the liquid surface geometries, whereby undesired refractions of the laser radiation can be reduced and possibly minimized. For example, nozzle geometries can be provided in a slot shape.

After the entrained particles in the liquid jet 14 have been treated by the laser beams 20a, 20b, the liquid jet 14 with the treated particles reaches a collection vessel 30. A liquid 32 with treated particles suspended therein collects in the collection vessel 30. The storage vessel 24 and the collection vessel 30 can be spaced apart from one another vertically, for example between 10 cm and 1 m.

In the present example, the storage vessel 24 is fluidically connected to the analysis device 23a. This makes it possible to supply liquid from the storage vessel 24 to the analysis device 23a. The fluidic connection 34 shown in FIG. 2 is shown only symbolically and also includes a possibility for returning the extracted liquid. In a correspondingly designed manner, the analysis device 23a is also connected to the collection vessel 30 via a further fluidic connection or line 36. This allows the analysis of the liquid 32 with the treated particles.

The analysis device 23a also comprises a measuring device for performing measurements on the liquid jet 14 itself, which is symbolically represented by the arrow with the reference number 33. The fluidic connection or line 34 connects the analysis device 23a to the storage vessel 24 in the present example. The line 34 can, however, also be connected directly to the beam generating device 28, for example. The further line 36 for the analysis device 23a, which in the present case connects the collection vessel 30 to the analysis device 23a, can alternatively, for example, also be connected to a discharge line 38 from the collection vessel 30. A discharge line 38 serves to discharge the liquid 32 from the collection vessel 30.

The discharge line 38 in the present example includes a flow divider device 40, via which a secondary flow 42 can be separated from a main flow 44 of the liquid 32 which is discharged from the collection vessel 30. This secondary flow 42 in the present example, is fed to an analysis device 23b, which can be provided in the device 10 in addition to or as an alternative to the analysis device 23a. After the liquid has been analyzed in the analysis device 23b, the secondary flow 42 is fed back to the main flow 44 and mixed therewith.

The main flow is then alternatively fed either to a drying device 46 or to a sterile filtration device 48, which comprises a filling device 48 for dispensing the suspension into a corresponding vessel 50.

The vessel 50 has an identification feature 52, which in the present case is designed as a QR code.

In addition to the discharge line 38 and the line 36, the collection vessel 30 is also fluidically connected to an extraction device 54. In the present case, it is possible to manually extract samples via the extraction device 54.

The two lasers 18a, 18b of the laser assembly 16 are arranged in FIG. 2 at the same height with respect to a flow direction of the liquid jet 14. The laser beams 20a, 20b hit the liquid jet 14 in a common area of incidence 55. The area of incidence 55 and a path of the laser beams 20a, 20b between the lasers 18a, 18b and the area of incidence 34 are within the housing 22.

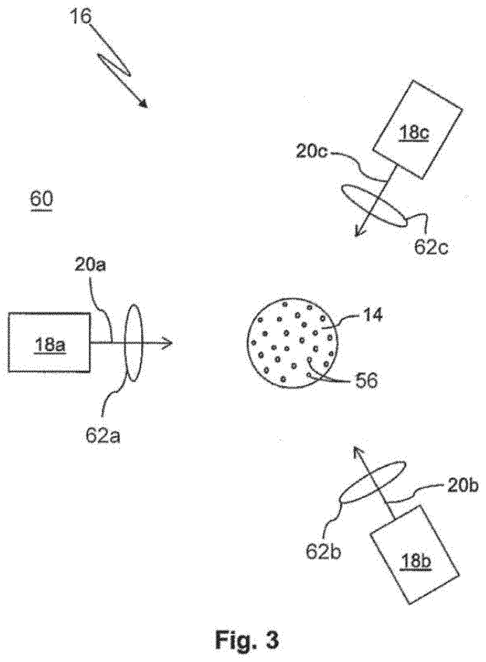

FIG. 3 shows an alternative laser assembly 16 with three lasers 18a, 18b, 18c for the treatment of particles 56 which are entrained in a liquid jet 14.

The lasers 18a, 18b, 18c are here arranged rotationally symmetrically with respect to the liquid jet 14 (in the present case at an angle of 120° to one another). The laser beams 20a-20c run in a common horizontal plane 60 (the plane of the drawing) perpendicular to the liquid jet 14.

In the present case, focusing devices 62, which in the present example are designed as lens optics 62a, 62b, 62c, are provided for focusing the laser beams 20a-20c on the liquid jet 14.

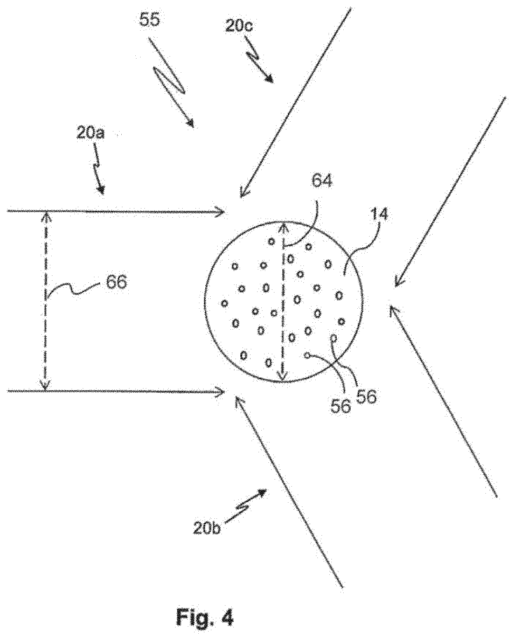

FIG. 4 shows an enlarged illustration of the area of incidence 55 of the laser beams 20a-20c on the liquid jet 14 during the treatment of the particles 56 with the laser assembly 16 according to FIG. 3. A diameter 64 of the liquid jet 14 is less than a width 66 of the laser beams 20a-20c.

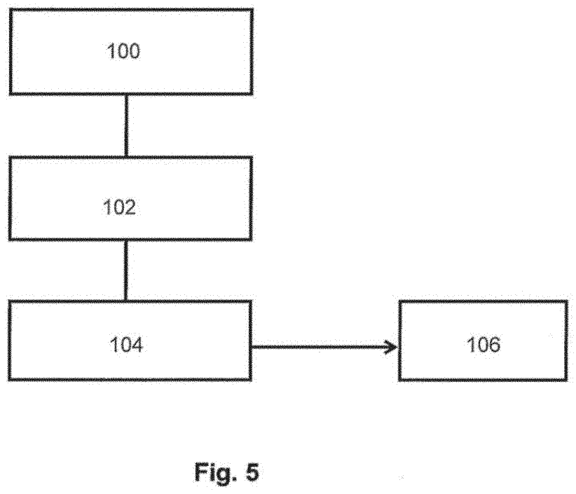

FIG. 5 shows a flow chart of a method according to the invention for treating particles. The method can be carried. out with the device 10 already described

In a first step 100, a liquid jet 14 is generated in which particles 38 are entrained.

In a step 102, the liquid jet 14 is irradiated with a plurality of, preferably pulsed, laser beams 20a-20c from different directions. The particles 38 in the liquid jet 14 are treated by the laser beams 20a -20c. One of the laser assemblies 16 described above can be used for this purpose.

In a step 104, the suspension is analyzed after the irradiation by means of the laser beams 20a-20c. The result of the analysis is transmitted to a database 106 in which it is stored in a manner that can be assigned to the appropriately treated or comminuted particles 56.

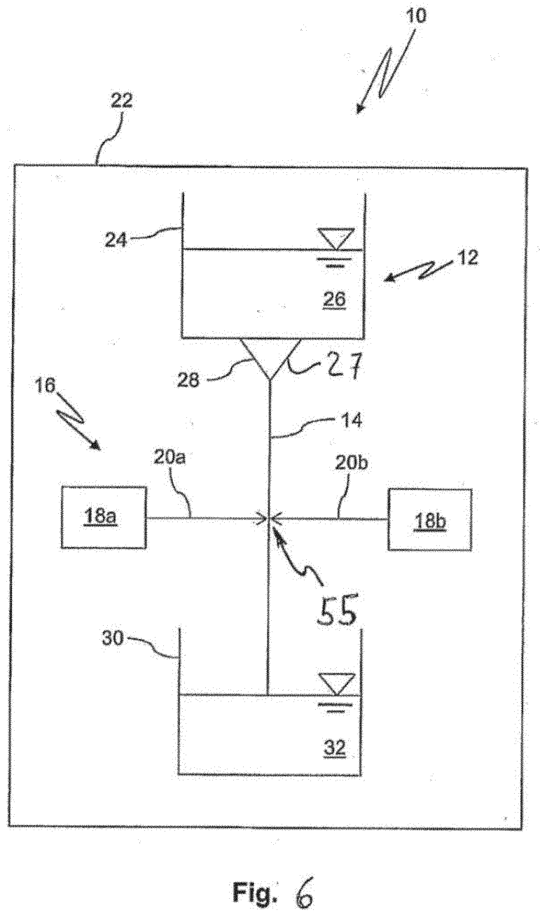

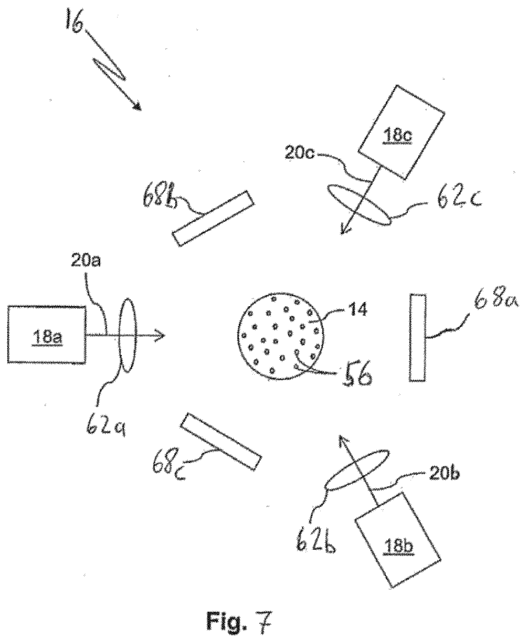

FIG. 6 shows a device 10 according to the invention for treating particles. The device 10 comprises a device 12 for generating a liquid jet 14 loaded with particles. The device 10 further comprises a laser assembly 16 with two lasers 18a, 18b. The lasers 18a, 18b emit pulsed laser beams 20a, 20b. The laser beams 20a, 20b are directed onto the liquid jet 14 from opposite directions. The laser assembly 16 and the device 12 are, as in FIG. 2, arranged together within a housing 22.

The device 12 comprises a storage vessel 24 in which a liquid 26, in this case an aqueous liquid 26, is stored with particles (not shown) suspended therein. A jet generating device 27 with a nozzle 28 is arranged on the storage vessel 24. The nozzle 28 works here without pressure, but it can also be pressure-operated. After exiting the nozzle 28, the liquid jet 14 drops down freely (unguided) under the influence of gravity.

After the entrained particles in the liquid jet 14 have been treated by the laser beams 20a, 20b, the liquid jet 14 with the treated particles reaches a collection vessel 30. A liquid 32 with treated particles suspended therein collects in the collection vessel 30. See also FIG. 2.

FIG. 3 shows a laser assembly 16 with three lasers 18a, 18b, 18c, similar to FIG. 3. This laser assembly 16 could be used in the device 10 according to FIG. 2 or 6 instead of the laser assembly 16 shown there.

The laser beams 20a-20c run here in a common horizontal plane 40 (the plane of the drawing) perpendicular to the liquid jet 14 and are arranged rotationally symmetrically with respect to the liquid jet 14. Two of the laser beams 20a-20c each enclose an angle of 120° between them.

In addition to an appropriate lens system 62a, 62b, 62c, a power measuring device 68a, 68b, 68c is arranged for each of the lasers 18a-18c. The power measuring devices 68a-68c determine the residual powers of the respective laser beans 20a-20c after they have interacted with the liquid jet 14 and the particles 38, in particular having treated the particles 38.

FIG. 5 shows a laser assembly 16 for a device 10 having precisely one laser 18 during the treatment of particles 56 which are entrained in a liquid jet 14.

The laser assembly 16 comprises a beam splitter device 72. The beam splitter device 72 divides the laser radiation emitted by the laser 18 into three separate laser beams 20a, 20b, 20c. The laser assembly 16 further comprises three light guide devices 70a, 70b, 70c. The light guide devices 70a-70c guide the laser beams 20a-20c to the liquid jet 14. The light guide devices 70a-70c are designed here as glass fiber cables. To focus or shape the laser beams 20a-20c, exit optics (not shown in detail) can be provided on the light guide devices 10a-10c.

In the embodiment according to FIG. 5, the particles 56 are remelted (melted) and fused by the laser beams 20a-20c. A wavelength of the laser beams 20a-20c is 343 nm here. A pulse repetition rate of the laser beams 20a-20c may be 100 Hz or more. A pulse duration of the light emission can be 10 nanoseconds. The laser beams 20a-20c can each have a fluence of at least 0.5 J/cm2. The aqueous liquid 26 can contain an inorganic oxidizing agent. The particles 38 can consist of gold or platinum.

FIG. 9 shows a flow chart of a method according to the invention for treating particles. The method can be carried out with the devices 10 described above.

In a first step, 100, a liquid jet 14 is generated in which particles 38 are entrained. A device 12 according to FIG. 2 can be used for this purpose.

Then, in a step 102, the liquid jet 14 is irradiated with a plurality of, preferably pulsed, laser beams 20a-20c from different directions. The particles 38 in the liquid jet 14 are treated by the laser beams 20a-20c. One of the laser assemblies 16 described above can be used for this purpose.

In a first step 100, a liquid jet. 14 is generated in which particles 38 are entrained.

In a step 102, the liquid jet 14 is irradiated with a plurality of, preferably pulsed, laser beams 20a-20c from different directions. The particles 38 in the liquid jet 14 are treated by the laser beams 20a-20c. One of the laser assemblies 16 described above can be used for this purpose.

In a step 103, the liquid 32 (with treated particles) of the liquid jet 14 is collected in a collection vessel 30.

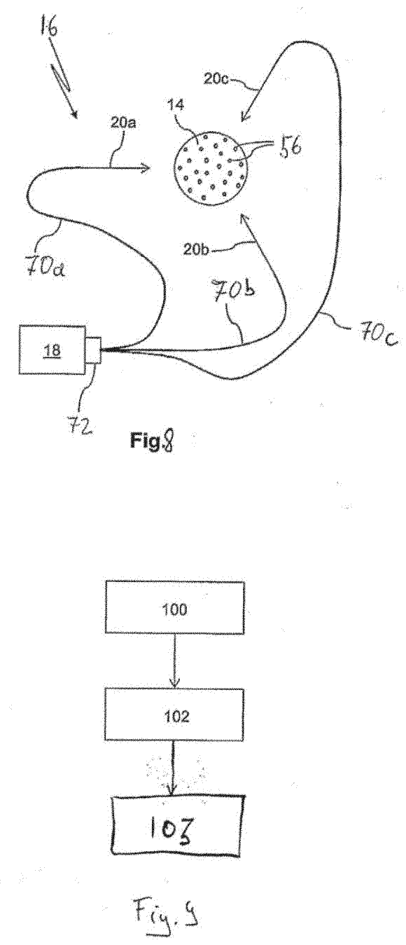

FIGS. 10, 11 and 12 illustrate the use of a reflection housing 74. The device 10 can include a reflection housing 74, in particular in the region of the area of incidence 55. In FIG. 10, the arrangement of a reflection housing 74 around the area of incidence 55 of a single laser beam 20 is shown. Further laser beams 20 are provided in planes that are offset from this along the flow direction 76 of the beam.

The reflection housing 74 is arranged in particular around the area of incidence 55, in particular over its entire circumference.

The reflection housing 74 has a reflective inner surface 78, that is to say facing the liquid jet. The (inner, liquid-jet-facing) surface 78 is designed and arranged in particular in such a way that it reflects the laser radiation which passes through the liquid jet 14 back into the liquid jet 14. For example, the inner surface 78 of the reflection housing 74 can be designed to have a circular arrangement concentric to the liquid jet 14. It can be provided that the reflection housing 74 has a plurality of lenses 80 (for example cylinder lenses) for coupling the individual laser beams 20 into the reflection housing 74. The lenses 80 are typically arranged and designed in such a way that they further direct the laser beam 20 onto the liquid jet 14 in she same direction in which it hits the lens 80.

The reflection on the inner surface 78 is shown in FIGS. 10 to 12, in each case by corresponding arrow indicators 82.

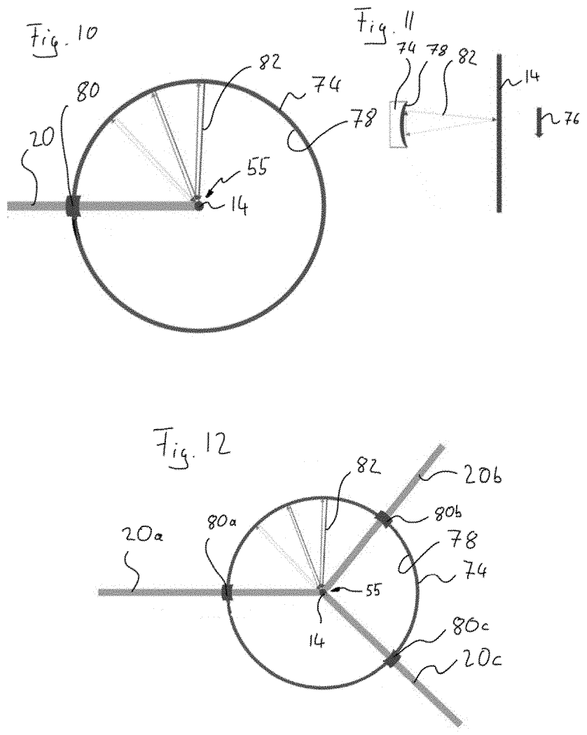

In FIG. 12, a variant is shown in which three laser beams 20a, 20b and 20c are coupled in a plane through corresponding lenses 80a, 80b and 80c in a reflection housing 74 and there hit the liquid jet 14 and are correspondingly reflected on the inner surface 78 of the reflection housing 74.

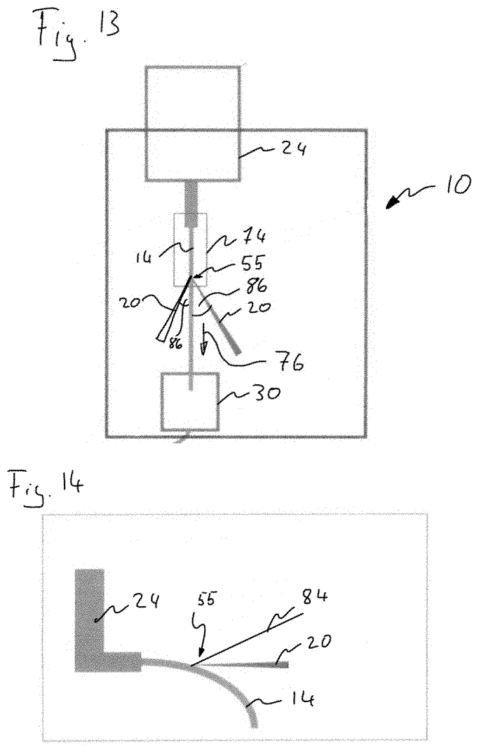

The coupling does not necessarily have to be accomplished using lenses 80. It can also be provided that the laser beams 20 are directed onto the beam 14 at an angle that runs obliquely to the direction of flow 76, so that they can be introduced into the reflection housing 74 from below or from above, for example, as shown in FIG. 13.

The lasers 20 of the laser assembly or when the method is being carried out can in particular be directed at an angle to the flow direction 76 of the liquid jet 14 which is less than or equal to the Brewster angle. Brewster's angle is represented by line 84 in FIG. 14. It can be provided that, depending on the type of radiation used and the optical properties of the phase boundaries between liquid jet 14 and the surrounding air, an angle of incidence 86 is selected at which the reflection is minimized when the laser beam 20 hits she liquid jet 14 and, when the laser beam 20 passes through, the transmission at the phase boundary between she liquid jet 14 and the surrounding air is minimized when exiting. In FIG. 14 a further laser is provided which radiates onto the liquid jet 14 from a different direction, but this is not shown.

In addition to the following claims, the following aspects can also define inventions that are to be understood as possible in combination with the further developments mentioned in the description. The individual features mentioned in the aspects are also to be understood as possible further developments of the inventions described in the description and the claims.

Aspects:

A method for treating particles comprising the steps of:
a) generating a liquid jet in which the particles are entrained,
b) irradiating the liquid jet with at least two, in particular pulsed, laser beams from different directions.

2. The method according to aspect 2, characterized in that the liquid jet in step b) is irradiated with at least three, in particular pulsed, laser beams from different directions in each case.

3. The method according to aspect 1 or 2, characterized in that the laser beams are rotationally symmetrical with respect to the liquid jet.

4. The method according to any of the preceding aspects, characterized in that the laser beams hit the liquid jet at the same height in she flow direction of the liquid jet.

5. The method according to any of the preceding aspects, characterized in that the laser beams run in a common plane.

6. The method according so any of the preceding aspects, characterized in that she particles are comminuted in step b).

7. The method according to aspect 6, characterized in that a pulse duration of the laser beams is in the picosecond range.

8. The method according to aspect 6 or 7, characterized in that a wavelength of the laser beams is at least 500 nm, preferably at least 520 nm, particularly preferably at least 530 nm, and/or that the wavelength of the laser beams is at most 560 nm, preferably at most 540 nm, particularly preferably at most 535 nm, very particularly preferably that the wavelength of she laser beams is 532 nm.

9. The method according to any of aspects 1 to 5, characterized in that the particles are remelted and/or fused in step b).

10. The method according to aspect 9, characterized in that a pulse duration of the laser beams is in the nanosecond range.

11. The method according to any of aspects 9 or 10, characterized in that a wavelength of the laser beams is at most 380 nm, preferably at most 360 nm, particularly preferably at most 350 nm, and/or that the wavelength of the laser beams is at least 310 nm, preferably at least 330 nm, particularly preferably at least 340 nm, very particularly preferably that the wavelength of the laser beams is 343 nm.

12. The method according to any of the preceding aspects, characterized in that the liquid jet falls freely downward under the influence of gravity.

13. A device for treating particles having

a device for generating a liquid jet loaded with particles,
a laser assembly for generating at least two, in particular pulsed, laser beams,
wherein the laser assembly is configured to direct the at least two laser beams onto the liquid jet from different directions.

14. The device according to aspect 13 further comprising an enclosure which is impermeable to the laser radiation of the laser beams and which surrounds an area of incidence of the laser beams on the liquid jet.

15. The device according to aspect 13 or 14, characterized in that the laser assembly comprises at least two, preferably three, lasers.

16. The device according to aspect 13 or 14, characterized in that the laser assembly has exactly one laser, one beam splitter device for generating the at least two laser beams and at least two light guide devices for guiding the at least two laser beams.

17. The device according to any of aspects 13 to 16, further comprising at least one power measuring device for measuring a residual power of at least one of the laser beams on the other side of the liquid jet.

1-23. (canceled)

24. A method for treating particles in a suspension, the method comprising the following steps:
a) generating a liquid jet in which the particles are entrained;
b) irradiating the liquid jet with at least two laser beams from mutually different directions in order to comminute the particles;
c) analyzing the suspension before and/or after irradiating the liquid jet with the at least two laser beams; and
d) collecting the liquid of the liquid jet in a collection vessel.

25. The method according to claim 24, wherein the step of analyzing the suspension comprises at least one process selected from the group consisting of a particle size measurement, an x-ray diffraction measurement, and a chromatographic measurement.

26. The method according to claim 24, which comprises carrying out an analysis before and after the irradiation and thereby recording the same measured variable and using the same measurement method.

27. The method according to claim 24, which comprises collecting the liquid jet in batches and assigning an analysis result to each batch.

28. The method according to claim 24, which comprises storing a result of the analysis in a database, and optionally assigning the result of the analysis to be stored in the database to each batch, and optionally storing the analysis results in at least one blockchain.

29. The method according to claim 24, wherein the analyzing step comprises an on-line and/or in-line measurement.

30. The method according to claim 24, wherein the analyzing step comprises a batch-wise measurement, wherein the batch-wise measurement is carried out for each batch.

31. The method according to claim 24, which comprises dividing the liquid of the liquid jet into a main flow and a secondary flow, carrying out the analyzing step on the liquid of the secondary flow, and mixing the secondary flow with the main flow following the analysis.

32. The method according to claim 24, further comprising a sterile filtration step, in which the liquid of the liquid jet is aseptically filled into a sealable, vessel.

33. The method according to claim 24, further comprising a spray-drying or freeze-drying step in which the particles present as a suspension in the liquid are converted into powder form.

34. The method according to claim 24, which comprises comparing the analysis results to a target variable or a previous analysis result and adjusting an irradiation in step b) based on a result of the comparison, if necessary.

35. The method according to claim 34, wherein the adjusting step comprises adjusting at least one of a pulse duration of a pulsed laser beam or a laser power of the laser beams.

36. A device for treating particles, the device comprising:
a jet generating device for generating from a suspension a liquid jet loaded with particles;
a laser assembly for generating at least two laser beams, said laser assembly being configured to direct the at least two laser beams onto the liquid jet from mutually different directions and to thereby irradiate all segments of an entire cross-section of the liquid jet;
a collection vessel configured and arranged to collect the liquid of the liquid jet after irradiation; and
an optional analysis device configured to analyze the suspension before and/or after irradiation of the liquid jet by the laser beams.

37. The device according to claim 36, wherein the analysis device is a device selected from the group consisting of a particle size measuring device, an x-ray diffraction measuring device, and a chromatographic measuring device.

38. The device according to claim 36, further comprising a flow divider configured to divide a liquid of the liquid jet into a main flow and a secondary flow, wherein the secondary flow is fed to said analysis device for analysis and the secondary flow is merged again with the main flow following the analysis.

39. The device according to claim 36, further comprising a portioning device configured to portion a liquid of the liquid jet into batches and to fluidically separate the batches from one another.

40. The device according to claim 36, further comprising an extraction device configured to extract sample volumes of the liquid from the liquid jet.

41. The device according to claim 36, further comprising a sterile filtration device for aseptic dispensing of the liquid from the liquid jet into a sealable vessel.

42. The device according to claim 36, further comprising a drying device selected from the group consisting of a spray-drying device and a freeze-drying device and configured to convert the particles in the suspension into powder form.

43. A pharmaceutical product, comprising:
nanoparticles with an active pharmaceutical ingredient;
said nanoparticles having been fragmented from particles in a suspension by the method according to claim 24; and
said nanoparticles being assigned an analysis result obtained by the step of analyzing the suspension before and/or after irradiating the liquid jet with laser beams.

44. The pharmaceutical product according to claim 43, wherein said nanoparticles consist of the active pharmaceutical ingredient.

45. The pharmaceutical product according to claim 43, wherein said nanoparticles are present in the form of a batch and were fragmented under uniform process conditions in step b) of claim 24, the nanoparticles being assigned a data record which includes the analysis result and at least one operating parameter that is characteristic for the irradiation in step b) of claim 24.

46. The pharmaceutical product according to claim 45, wherein the batch of nanoparticles is in a mechanically manageable vessel, and the vessel comprises a machine-readable identification feature which uniquely assigns the data record to the vessel, and wherein the nanoparticles have been transferred into the vessel using sterile filtration.

47. The pharmaceutical product according to claim 43, wherein the nanoparticles are present in a suspension in an aqueous medium, the suspension comprising an additive for particle stabilization selected from the group consisting of cellulose, polyvinyl alcohol, polyvinylpyrrolidone, and sodium dodecyl sulfate or another surface-active substance.

48. The pharmaceutical product according to claim 43, wherein the particles have been converted into powder form using a spray drying or freeze-drying step.
